(19) United States
(12) Patent Application Publication
(10) Pub. No.: US 2025/0352537 A1
(43) Pub. Date: Nov. 20, 2025

(54) PHARMACEUTICAL COMPOSITIONS OF TRICYCLIC AKR1C3 DEPENDENT KARS INHIBITOR AND METHODS FOR MAKING SAME

(71) Applicant: NOVARTIS AG, Basel (CH)

(72) Inventors: Oliver Graner, Basel (CH); Cornelius Stephan Harlacher, Reinach (CH)

(21) Appl. No.: 18/871,848

(22) PCT Filed: Jul. 20, 2023
(86) PCT No.: PCT/IB2023/057404

§ 371 Date: Dec. 5, 2024

Related U.S. Application Data
(60) Provisional application No. 63/369,407, filed on Jul. 26, 2022.

(51) Int. Cl. A61K 31/4747; A61K 9/16; A61K 9/20; A61K 9/28; A61K 9/48; A61K 9/51

(52) U.S. Cl. CPC A61K 31/4747; A61K 9/1611; A61K 9/1617; A61K 9/1623; A61K 9/1635; A61K 9/1641; A61K 9/1652; A61K 9/1694; A61K 9/2009; A61K 9/2013; A61K 9/2018; A61K 9/2027; A61K 9/2054; A61K 9/28; A61K 9/485; A61K 9/4858; A61K 9/4866; A61K 9/5115; A61K 9/5123; A61K 9/5138; A61K 9/5161; A61K 9/5192

(57) ABSTRACT

The present invention relates to solid phase pharmaceutical compositions of 6′-fluoro-N-(4-fluorobenzyl)-4′-oxo-3′,4′-dihydro-1′H-spiro[piperidine-4,2′-quinoline]-1-carboxamide that is useful as a AKR1C3 dependent KARS inhibitor. The present invention also relates to processes for the preparation of said pharmaceutical compositions of said compound, methods of using said pharmaceutical compositions in the treatment of various diseases and disorders, and their use in diseases and disorders mediated by an AKR1C3 dependent KARS inhibitor.

### FIELD OF THE INVENTION

The present invention relates to pharmaceutical compositions of 6′-fluoro-N-(4-fluorobenzyl)-4′-oxo-3′,4′-dihydro-1′H-spiro[piperidine-4,2′-quinoline]-1-carboxamide that is useful as a AKR1C3 dependent KARS inhibitor. The present invention also relates to processes for the preparation of said pharmaceutical compositions of said compound, methods of using said pharmaceutical compositions in the treatment of various diseases and disorders, and their use in diseases and disorders mediated by an AKR1C3 dependent KARS inhibitor.

### BACKGROUND TO THE INVENTION

The NFE2L2/NRF2-KEAP1 pathway has a strong genetic basis in cancer. The TCGA sequencing effort reported that this pathway was altered in 34% of lung squamous cell carcinomas (Hammerman P S et al. Comprehensive genomic characterization of squamous cell lung cancers. Nature 489, 519-525 (2012)). In addition, TCGA and other groups have reported significant mutation of this pathway in other solid tumor indications, including head and neck squamous cell carcinoma and hepatocellular carcinoma. Aberrant activation of the NRF2 pathway can occur by gain of function genetic alterations in NRF2 or loss of function genetic alterations in KEAP1 or CUL3 that lead to stabilization of NRF2 and elevated expression of its target genes. The uncontrolled transcription of those target genes confers advantages to cancer cells such as malignancy and protection against oxidative stress, chemotherapy and radiotherapy (Jaramillo M C, Zhang D D. The emerging role of the Nrf2-Keap1 signaling pathway in cancer Genes Dev. 27, 2179-2191 (2013)). Exacerbated NRF2 activity in tumors has been associated with poor prognosis (Shibata T, Ohta T, Tong K I, Kokubu A, Odogawa R, Tsuta K, Asamura H, Yamamoto M, Hirohashi S. Cancer related mutations in NRF2 impair its recognition by Keap1-Cul3 E3 ligase and promote malignancy. Proc Nat!Acad Sci USA 105, 13568-13573 (2008)). To the best of our knowledge, there is currently no approved therapy to selectively target cancers with genetic alterations on the NRF2/KEAP1 pathway, which thus represents an unmet medical need.

Aldehyde keto reductase 1C3 (AKR1C3) is one of the numerous target genes of the transcription factor NRF2, whose expression is upregulated in NRF2/KEAP1 mutated cancers (MacLeod AK, Acosta-Jimenez L, Coates P J, McMahon M, Carey F A, Honda T, Henderson C J and Wolf C R. Aldo-keto reductases are biomarkers of NRF2 activity and are coordinately overexpressed in non-small cell lung cancer. Br J Cancer 115, 1530-1539 (2016)). AKR1C3 (also named type 2 3a(17p)-hydroxysteroid dehydrogenase) is an NADP(H)-dependent ketosteroid reductase, member of the aldo-keto reductase (AKR) superfamily, that plays a role in steroid hormone metabolism and signaling, as well as xenobiotic detoxification. Some known substrates for AKR1C3 are the endogenous substrates 5α-dihydrotestosterone, A4-androstene-3,17-dione and progesterone (Penning T M, Burczynski M E, Jez J M, Hung C F, Lin H K, Ma H, Moore M, Palackal N, Ratnam K. Human 3α-hydroxysteroid dehydrogenase isoforms (AKR1C1-AKR1C4) of the aldo-keto reductase superfamily: functional plasticity and tissue distribution reveals roles in the inactivation and formation of male and female sex hormones. Biochem. J. 351, 67-77 (2000)), as well as the synthetic prodrugs coumberone (Halim M, Yee D J, Sames D. Imaging Induction of Cytoprotective Enzymes in Intact Human Cells: Coumberone, a Metabolic Reporter for Human AKR1C Enzymes Reveals Activation by Panaxytriol, an Active Component of Red Ginseng J. Am. Chem. Soc.130, 14123-14128 (2008)), PR104 (Jamieson S M, Gu Y, Manesh D M, EI-Hoss J, Jing D, Mackenzie K L, Guise C P, Foehrenbacher A, Pullen S M, Benito J, Smaill J B, Patterson A V, Mulaw M A, Konopleva M, Bohlander S K, Lock R B, Wilson W R. A novel fluorometric assay for aldo-keto reductase 1C3 predicts metabolic activation of the nitrogen mustard prodrug PR-104A in human leukaemia cells. Biochem Pharmacol. 88, 36-45 (2014)) and TH3424/0B13424 (Threshold pharmaceuticals WO 2016/145092 A1). We report the identification of tricyclic ketone compounds that get converted to lysine t-RNA synthetase (KARS) inhibitors by AKR1C3 in the presence of NADPH. Lysine t-RNA synthetase is a ubiquitous enzyme essential for protein synthesis that is part of the multi-tRNA synthetase complex.

AKR1C3 dependent KARS inhibitors provide an attractive strategy to selectively treat tumors that overexpress AKR1C3 compared to normal tissues, such as NRF2/KEAP1 mutated cancers and other types of cancers reported to overexpress AKR1C3 (Guise C P, Abbattista M R, Singleton R S, Holford S D, Connolly J, Dachs G U, Fox S B, Pollock R, Harvey J, Guilford P, Donate F, Wilson W R, Patterson A V. The bioreductive prodrug PR-104A is activated under aerobic conditions by human aldo-keto reductase 1C3. Cancer Res.70,1573-1584 (2010)) such as breast cancers (Lewis M J, Wiebe J P, Heathcote J G. Expression of progesterone metabolizing enzyme genes (AKR1C1, AKR1C2, AKR1C3, SRD5A1, SRD5A2) is altered in human breast carcinoma. BMC Cancer4, 27 (2004)) and prostate cancers (Fung K M, Samara ENS, Wong C, Metwalli A, Krlin R, Bane B, Liu CZ, et al. Increased expression of type 2 3α-hydroxysteroid dehydrogenase/type 5 17β-hydroxysteroid dehydrogenase (AKR1C3) and its relationship with androgen receptor in prostate carcinoma. Endocr Relat Cancer 13,169-180 (2006)).

6′-fluoro-N-(4-fluorobenzyl)-4′-oxo-3′,4′-dihydro-1′H-spiro[piperidine-4,2′-quinoline]-1-carboxamide, first disclosed in WO/2021/005586, is a selective AKR1C3 reductase dependent KARS inhibitor. There remains a need in the art for novel composition for delivering AKR1C3 reductase KARS inhibitors an methods for treating selective AKR1C3 reductase dependent KARS inhibitor assocaited diseases using the same, which is stable and provides optimum bioavailability.

### SUMMARY OF THE INVENTION

It has now been found that pharmaceutical composition of the present disclosure, and compositions thereof, are useful for administering a selective AKR1C3 inhibitor to a patient in need thereof and exhibit desireable characteristics for the same. In general, the pharmaceutically aceptable compositions disclosed herein are useful for treating or lessening the severity of a varitey of diseases or disorders, as described in detail herein.

### BRIEF DESCRIPTION OF THE FIGURES

FIG. 1 is a an illustration of Compound (I) desupersaturation profiles in FaSSIF-V1 when added as DMSO solution at drug loads from 100 to 2000 ppmw

FIG. 2: NSG Table Compaction Properties

FIG. 3: 2-step dissolution of HPMC-AS based ASG, FaSSGF →FaSSIF-V1 switch after 60 min, final drug load 100 ppmw

### DETAILED DESCRIPTION OF THE INVENTION

The present disclosure is based at least in part on the identification of a compounds that inhibits AKR1C3 and methods of use the same to treat AKR1C3 assocaited diseases. Dislcosed herein is Compound (I), and pharmaceutical compositions thereof'

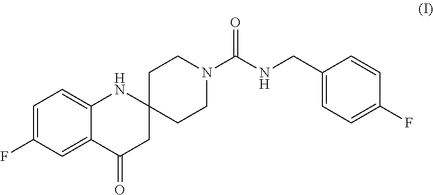 [SMILES: O=C1CC2(CCN(C(=O)NCc3ccc(F)cc3)CC2)Nc2ccc(F)cc21]

Compound of Formula (I), 6′-fluoro-N-(4-fluorobenzyl)-4′-oxo-3′,4′-dihydro-1′H-spiro[piperidine-4,2′-quinoline]-1-carboxamide, is actived in a variety of assays and therapeutic models, acting as a selective AKR1C3 inhibitor.

It would be desireable to provide pharmaceutically acceptable compositions comprising Compound (1) that imparts characteristics such as improved stability, improved oral bioavailablity, and low toxicity risk. Accordingly, the present disclosure provides pharmaceutical compositions of Compound (1).

In one aspect, the present invention provides a pharmaceutical composition of a compound represented by Formula (I)

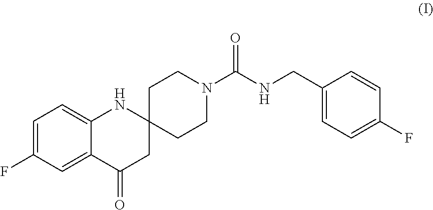 [SMILES: O=C1CC2(CCN(C(=O)NCc3ccc(F)cc3)CC2)Nc2ccc(F)cc21]

Comprising the Compound of Formula (I) stabilized in amorphous form with a polymer. Amorphous Spray Granules (ASG) Composition

In one aspect, the present invention provides a pharmaceutical compostion for oral administration of Compound (1) to a subject, wherein Compound (1) is formulated as amorphous spray granules. In some embodiments, the pharmaceutical composition of the present invention comprises:

A pharmaceutical composition of a compound represented by Formula (I)

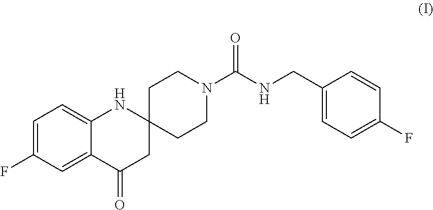 [SMILES: O=C1CC2(CCN(C(=O)NCc3ccc(F)cc3)CC2)Nc2ccc(F)cc21]

comprising:
(i) an intragranular blend, wherein the intragranular blend comprises:
   (a) an amorphous spray granulation comprising:
      (i) the compound of Formula (I), wherein the compound is present in amorphous form;
      (ii) a polymer;
   (b) a suspending agent;
   (c) a carrier;
(ii) an extragranular blend, wherein the extragranular blend comprises:
   (d) one or more fillers;
   (e) a disintegrant;
   (f) a glidant; and
   (g) a lubricant.

### Nano Spray Granules (NSG) Composition

In one aspect, the present invention provides a pharmaceutical compostion for oral administration of Compound (1) to a subject, wherein Compound (1) is formulated as a nano spray granulation. In some embodiments, the pharmaceutical composition of the compound represented by Formula (I)

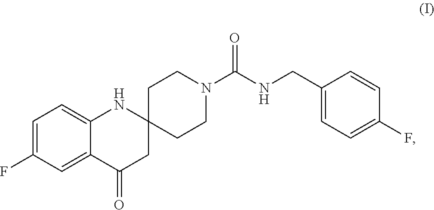 [SMILES: O=C1CC2(CCN(C(=O)NCc3ccc(F)cc3)CC2)Nc2ccc(F)cc21]

of the present invention comprises:
(i) a nanosized crystalline spray granulate, comprising:
   (a) the compound of Formula (I), in crystalline Form A, wherein the crystals have been nano sized,
   (b) a polymer,
   (c) a surfactant;
   (d) a carrier;
(ii) an extragranular blend, wherein the extragranular blend comprises:
   (e) one or more fillers;
   (f) a disintegrant;
   (g) a glidant; and
   (h) a lubricant.

### A. Compound (1)

As defined above, a pharmaceutical compoisiton of the present invention is an amorphous spray granulation or nano spray granulation comprising Compound (1). Compound (1) can be prepared according to Example 40 of WO/2021/005586, which is incorporated by reference herein.

In some embodiments, the crystalline solid for fo Compound (1) is anhydrous Form A of Compound (1). In some embodiments, Form A of Compound (1) is a form having at least 1, 2, 3, 4, or 5 X-ray powder diffraction peaks listed in Table 1 below:

TABLE 1
XRPD Peak positions for From A of Compound (I)

| 2θ [°] | d value [Å] | Intensity [%] |
|---|---|---|
| 9.57 | 9.239 | 100 |
| 10.45 | 8.458 | 19 |
| 13.35 | 6.629 | 19 |
| 15.74 | 5.625 | 16 |
| 17.11 | 5.178 | 32 |
| 19.17 | 4.626 | 79 |
| 21.01 | 4.225 | 40 |
| 22.42 | 3.962 | 11 |
| 27.25 | 3.270 | 11 |
| 30.42 | 2.936 | 21 |
| 31.69 | 2.821 | 13 |

In another aspect of the above embodiment, the crystalline Form A of compound of Formula (I) is characterized by a x-ray powder diffraction pattern comprising two or more 2θvalues selected from the group consisting of 9.6±0.2° 2θ, 10.5±0.2° 2θ, 13.4±0.2° 2θ, 15.7±0.2° 2θ, 17.1±0.2° 2θ, 19.2 0.2° 2θ, 21.0±0.2° 2θ, 22.4 0.2° 2θ, 27.3±0.2° 2θ, 30.4 0.2° 2θand 31.7±0.2° 2θ, measured at a temperature of about 25° C. In another aspect of the above embodiment, the crystalline Form A of compound of Formula (I) is characterized by a x-ray powder diffraction pattern comprising three or more 20 values (CuKα λ=1.54184 Å) selected from the group consisting of 9.6±0.2° 2θ, 10.5±0.2° 2θ, 13.4±0.2° 2θ, 15.7±0.2 ° 2θ, 17.1±0.2° 2θ, 19.2±0.2° 2θ, 21.0±0.2° 2θ, 22.4±0.2° 2θ, 27.3±0.2° 2θ, 30.4±0.2 ° 2θand 31.7±0.2° 2θmeasured at a temperature of about 25° C. In another aspect of the above embodiment, the crystalline Form A of compound of Formula (I) is characterized by a x-ray powder diffraction pattern comprising four or more 26 values selected from the group consisting of 9.6±0.2° 2θ, 10.5±0.2° 2θ, 13.4±0.2° 2θ, 15.7±0.2° 2θ, 17.1±0.2° 2θ, 19.2±0.2° 2θ, 21.0±0.2° 2θ, 22.4 0.2° 2θ, 27.3±0.2° 2θ, 30.4±0.2° 2θand 31.7±0.2° 2θmeasured at a temperature of about 25° C. In another aspect of the above embodiment, the crystalline Form A of compound of Formula (I) is characterized by a x-ray powder diffraction pattern comprising five or more 26 values selected from the group consisting of 9.6±0.2° 2θ, 10.5±0.2° 2θ, 13.4 0.2° 2θ, 15.7±0.2° 2θ, 17.1±0.2° 2θ, 19.2±0.2° 2θ, 21.0 0.2° 2θ, 22.4±0.2° 2θ, 27.3±0.2° 2θ, 30.4±0.2° 2θand 31.7±0.2° 2θmeasured at a temperature of about 25° C.

In some embodiments, Compound (I) is present in the pharmaceutical composition in an amount from about 1 wt % to about 40 wt %. In some embodiments, Compound (I) is present in the pharmaceutical composition in an amount from about 5 wt % to about 20 wt %. In some embodiments, Compound (I) is present in the pharmaceutical composition in an amount from about 8 wt % to about 14 wt %. In some embodiments, Compound (I) is present in the pharmaceutical composition in an amount from about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt % about 19 wt %, or about 20 wt %. In some embodiments, Compound (I) is present in the pharmaceutical composition in an amount of about 11.9 wt %. In another embodiment, Compound (I) is present in an amount of about 12.5%. In further embodiments, Compound (I) is present in amount of about 20 to 40 wt %. In some embodiments, Compound (I) is present in an amount of about 40 wt %.

Particle Size Distribution for Nanosuspension granulate

The granule particle size of the nanosuspension granulate is measured, for example, by laser diffraction methodology (e.g. particle size distribution (PSD)) using methods and instruments known to the skilled person in the art.

According to the present invention, Compound (I) can be used directly or can be subjected to mechanical means to reduce the average particle size to less than 1000 nm.

The particle size is measured, for example, by laser diffraction methodology (e.g. particle size distribution (PSD)) using methods and instruments known to the skilled person in the art. Preferably, the particle size as measured by PCS is less than 500 nm, more preferably less than 350 nm and most preferably less than 250 nm. In one embodiment, the particle size of the suspension as measured by PCS is between about 50 nm to about 1000 nm, or between about 50 nm to 500 nm, or between about 50 nm to about 350 nm, or between about 100 nm to 170 nm, e.g. the particle size is about 50 nm, or about 70 nm, or about 90 nm, or about 100 nm, or about 110 nm, or about 120 nm, or about 130 nm, or about 140 nm, or about 150 nm, or about 160 nm, or about 170 nm, or about 180 nm, or about 190 nm, or about 200 nm, or about 230 nm or about 250 nm, or about 280 nm, or about 300 nm, or about 320 nm, or about 350 nm, or about 370 nm, or about 400 nm, or about 450 nm, or about 500 nm. More preferably, the particle size is between about 100 nm to about 350 nm, or between about 110 nm to about 180 nm, or between about 250 nm to about 350 nm. The particles formed are stabilized by the presence of a polymer in the intragranular blend, as defined herein, which is able to maintain the particles at the desired size, in a stable state.

API particles can be prepared by suitable milling techniques, e.g. those well known in the art such as, for example, jet milling, pin-milling, and wet-ball milling

### B. Polymer

As defined above, a pharmaceutical composition of the present invention is an amorphous spray or nano spray granulation comprising a polymer. In some embodiments, the polymer comprises an organic polymer. Suitable polymers include, but are not limited to, cellulose or starch, micro-crystalline cellulose (“MCC”), Avicel P H 101 (FMC BioPolymer), acacia, sodium alginate, gelatine, starch, pregeliatinised starch, methylcellulose, hydroxypropyl methylcellulose (“HPMC”), Hydroxypropyl methylcellulose acetate succinate (“HPMC-AS”), hydroxypropylcellulose, hydroxyethylcellulose, polyethylene glycol, polyvinylpyrrolidone (“PVP”), polyvnyl acetate phthalate (“PVAP”), copolyvidone (e.g. Kollidon® VA 64), crospovidon (e.g. Kollidon® CL), carrageenan, such as Gelcarin G P 812 ethylcellulose and cellulose acetate or polyacrylates, e.g. ammonio methacrylate copolymers (Eudragit RS/RL), METHACRYLIC ACID-ETHYL ACRYLATE COPOLYMER (Eudragit L100-55) polyvinylacetate, polyvinyl caprolactam-polyvinyl acetate-polyethylene glycol graft copolymer (Soluplus®), or combinations thereof.

In some embodiments, the polymer comprises hydroxypropyl methylcellulose (“HPMC”). In some further embodiments, the polymer comprises Hydroxypropyl methylcellulose acetate succinate (“HPMC-AS”).

In some embodiments, the polymer comprises polyvinylpyrrolidone (“PVP”). In some further embodiments, the polymer comprises polyvinylpyrrolidone 30 (“PVP-30”).

In some embodiments, the polymer is any amorphous carrier commonly utilized in the formulation of pharmaceutical compositions for oral administration.

In some embodiments, the polymer is present in the pharmaceutical composition in an amount from about 1 wt % to about 40 wt %. In some embodiments, the polymer is present in the pharmaceutical composition in an amount from about 15 wt % to about 30 wt %. In some embodiments, the polymer is present in the pharmaceutical composition in an amount from about 22 wt % to about 28 wt %. In some embodiments, the polymer is present in the pharmaceutical composition in an amount from about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt % about 19 wt %, or about 20 wt %. In some embodiments, the polymer is present in the pharmaceutical composition in an amount about 8.34 wt %. In some embodiments, the polymer is present in the pharmaceutical composition in an amount about 16.67 wt %. In some embodiments, the polymer is present in the pharmaceutical composition in an amount about 26.6 wt %.

### C. Suspending Agent

As defined above, a pharmaceutical composition of the present invention is an amorphous spray or nano spray granulation comprising a suspending agent. In some embodiments, the suspending agent is any suspending commonly utilized in the formulation of pharmaceutical compositions for oral administration. In some embodiments, the pharmaceutical composition of the present invention comprises a suspending agent selected from: simethicone, Silicon Dioxide, silica, colloidal silica, magnesium silicate, magnesium trisilicate, talc and other forms of silica such as aggregated silicate and hydrated silica. In further embodiments, the suspending agent is silicon dioxide.

### D. Carrier

As defined above, a pharmaceutical composition of the present invention is an amorphous spray or nano spray granulation comprising a carrier.

In some embodiments, the carrier is any arrier commonly utilized in the formulation of pharmaceutical compositions for oral administration. In some embodiments, the pharmaceutical composition of the present invention comprises a carrier selected from: lactose, dextrose, sucrose, mannitol, sorbitol, cellulose including silicified microcrystalline cellulose, sodium saccharin, glucose and/orglycine. Furthermore, in addition to those listed above, the tablet and/or capsule diluent that are suitable in the disclosure include but not limiting calcium carbonate, calcium hydrogen phosphate, calciumphosphate, calcium sulfate, cellulose powder, glucan binding agent, fructose, kaolin, starch, pregelatinized starch, compressible sugar and confectionery sugar and combinations thereof. In a further embodiment, the carrier is selected from lactose or mannitol and combinations thereof.

### E. Filler

As defined above, a pharmaceutical composition of the present invention is an amorphous spray or nano spray granulation comprising at least one filler.

In some embodiments, the filler is any filler commonly utilized in the formulation of pharmaceutical compositions for oral administration. In some embodiments, the pharmaceutical composition of the present invention comprises a filler selected from: cellulose derivatives such as microcrystalline cellulose or lignocellulose (including microcrystalline cellulose and silicified microcrystalline cellulose), lactose, anhydrous lactose or lactose monohydrate, sucrose, starch, pregelatinized starch, low substituted hydroxypropyl cellulose (L-HPC), dextrose, mannitol (including mannitol Pearlitol SD 200), fructose, xylitol, sorbitol, corn starch, modified corn starch, inorganic salts such as calcium carbonate, calcium phosphate, dicalcium phosphate, calcium sulfate, dextrin/glucose binder, maltodextrin, compressible sugar and other known compatibilizers or fillers/or mixtures of two or more of them.

### F. Disintegrant

As defined above, a pharmaceutical composition of the present invention is an amorphous spray or nano spray granulation comprising a disintegrant.

In some embodiments, the disintegrant is any disintegrant commonly utilized in the formulation of pharmaceutical compositions for oral administration. In some embodiments, the pharmaceutical composition of the present invention comprises a disintegrant selected from: croscarmellose sodium, crospovidone, starch, potato starch, pregelatinized starch, corn starch, sodiumcarboxymethyl starch, sodium starch glycolate, microcrystallinecellulose, low substituted hydroxypropyl cellulose (L-HPC), sodium carboxymethyl cellulose and other known disintegrants. Several specific types of disintegrants are suitable for use in the formulations described herein. Further, in addition to the above disintegrants, the disintegrant suitable for use in the tabletof the present disclosure includes, but is not limited to, alginic acid, polakolin potassium, sodium starch glycolate and pregelatinized starchand combinations thereof. In further embodiments the disintegrant is sodium carboxymetyl cellulose. In other embodiments, the disintegrant is low substituted hydroxypropyl cellulose (L-HPC).

### G. Glidant

As defined above, a pharmaceutical composition of the present invention is an amorphous spray or nano spray granulation comprising a glidant.

In some embodiments, the glidant is any glidant commonly utilized in the formulation of pharmaceutical compositions for oral administration. In some embodiments, the pharmaceutical composition of the present invention comprises a glidant selected from: silica, colloidal silica, magnesium silicate, magnesium trisilicate, talc and other forms of silica such as aggregated silicate and hydrated silica.

### H. Lubricant

As defined above, a pharmaceutical composition of the present invention is an amorphous spray or nano spray granulation comprising a lubricant.

In some embodiments, the lubricant is any lubricant commonly utilized in the formulation of pharmaceutical compositions for oral administration. In some embodiments, the pharmaceutical composition of the present invention comprises a lubricant selected from: magnesium stearate, zinc stearate, calcium stearate, talc, carnauba wax, stearic acid, palmitic acid, sodiumstearyl fumarate, sodium lauryl sulfate, glyceryl palmitostearate,palmitic acid, myristic acid and hydrogenation vegetable oil and fat andother known lubricant and/or mixtures of two or more of them. Further, in addition to the above-mentioned lubricants, the lubricants suitable for use in the tablet and/or capsule of the present disclosure includes, but is not limited to, glyceryl behenate, light mineral oil, polyethyleneglycol, hard-purified stearic acid, and combinations thereof.

### I. Surfactant

As defined above, a pharmaceutical composition of the present invention is an amorphous spray or nanospray granulation comprising a surfactant.

In some embodiments, the surfactant is any surfactant commonly utilized in the formulation of pharmaceutical compositions for oral administration. In some embodiments, the pharmaceutical composition of the present invention comprises a surfactant selected from: acacia, cholesterol, diethanolamine, glyceryl monostearate, lanolin alcohols, lecithin, mono-and di-glycerides, monoethanolamine, oleic acid, oleyl alcohol.poloxamer, polyoxyethylene 50 stearate, polyoxyethylene 35 castor oil, polyoxyethylene 40 hydrogenated castor oil, polyoxyl 10 oleyl ether,polyoxyethylene 20 cetostearyl ether, polyoxyethylene 40 stearate,polysorbate 20, polysorbate 40, polysorbate 60, polysorbate 80, propyleneglycol diacetate, propylene glycol monostearate, sodium lauryl sulfate, sodium stearate, sorbitan monolaurate, sorbitan monooleate, sorbitan monopalmitate, sorbitan monostearate, stearic acid, triethanolamine, emulsifying wax and the combinations thereof.

### Pharmaceutical Compositions

As described above, in some embodiments, the pharmaceutical composition is an amorphous spray granulate comprising:

### EMBODIMENTS

1. A pharmaceutical composition of a compound represented by Formula (I)

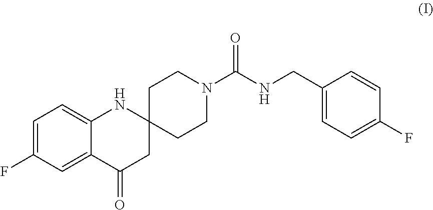 [SMILES: O=C1CC2(CCN(C(=O)NCc3ccc(F)cc3)CC2)Nc2ccc(F)cc21]

Comprising the Compound of Formula (I) stabilized in amorphous form with a polymer.
2. The pharmaeutical composition of claim 1, wherein the Compound of Formula (I) is present in about 5 to 80 wt %, about 10 to 50 wt %, about 25 to 40 wt %, or about 30 wt %.
3. The pharmaeutical composition of embodiment 1, wherein the polymer is selected from: hydroxypropyl methylcellulose, Hydroxypropyl methylcellulose acetate succinate (HPMC-AS), hydroxypropyl methylcellulose phtalate, hydroxypropyl cellulose, povidone (PVP), copovidone (PVP VA 64), cellulose acetate, cellulose acetate phtalate, or polyacrylates, e.g. ammonio methacrylate copolymers (e.g. Eudragit RS/RL), METHACRYLIC ACID-ETHYL ACRYLATE COPOLYMER (e.g. Eudragit L100 or L100-55), polyvinylacetate, polyvinylacetate phtalate, polyvinyl caprolactam-polyvinyl acetate-polyethylene glycol graft copolymer (Soluplus®).
4. The pharmaeutical composition of embodiment 3, wherein the polymer is HPMC-AS-L, HPMC-AS-M, HPMC-AS-H, or Eudragit L.
5. A pharmaceutical composition of a compound represented by Formula (I)

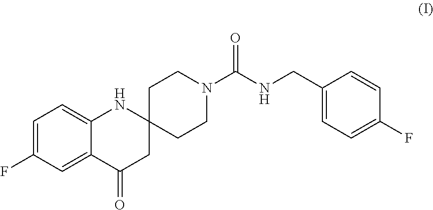 [SMILES: O=C1CC2(CCN(C(=O)NCc3ccc(F)cc3)CC2)Nc2ccc(F)cc21]

comprising:
(i) an intragranular blend, wherein the intragranular blend comprises:
   (a) an amorphous spray granulate comprising:
      (i) the compound of Formula (I), wherein the compound is present in amorphous form;
      (ii) a polymer;
   (b) a suspending agent;
   (c) a carrier;
(ii) an extragranular blend, wherein the extragranular blend comprises:
   (d) a filler;
   (e) disintegrant;
   (f) a glidant; and
   (g) a lubricant.
6. The pharmaceutical composition of embodiment 5 comprising:
   (i) an intragranular blend, wherein the intragranular blend comprises:
      (a) an amorphous spray granulate comprising:
         (i) the compound of Formula (I), wherein the compound is present in amorphous form, in an amount of about 5 wt % to 20 wt %;
         (ii) a polymer in an amount of about 10 wt % to 60 wt %;
      (b) a suspending agent in an amount of about 0.5 wt % to 2.0 wt %;
      (c) a carrier in an amount of about 20 wt % to 80 wt %;
   (ii) an extragranular blend, wherein the extragranular blend comprises:
      (d) a filler in an amount of about 10 wt % to 40 wt %;
      (e) a disintegrant in an amount of about 0 wt % to 5 wt %;
      (f) a glidant in an amount of about 0.5 wt % to 2.0 wt %; and
      (g) a lubricant in an amount of about 0.5 wt % to 3.0 wt %.
7. The pharmaceutical composition of embodiment 6 comprising:
   (i) an intragranular blend, wherein the intragranular blend comprises:
      (a) an amorphous spray granulate comprising:
         (i) the compound of Formula (I), wherein the compound is present in amorphous form, in an amount of 11.9 wt %;
         (ii) a polymer in an amount of about 26.6 wt %;;
      (b) a suspending agent in an amount of about 1.3 wt %;
      (c) a carrier in an amount of about 30.3 wt %;
   (ii) an extragranular blend, wherein the extragranular blend comprises:
      (d) a filler in an amount of about 24.6 wt %;
      (e) a disintegrant in an amount of about 2.8 wt %;
      (f) a glidant in an amount of about 1.0 wt %; and
      (g) a lubricant in an amount of about 1.5 wt %.
8. The pharmaceutical composition of any embodiments 5-7, wherein the polymer is hydroxypropyl methyl cellulose acetate succinate (HPMC-AS).
9. The pharmaceutical composition of embodiment 8, wherein the hydroxypropyl methyl cellulose acetate succinate is selected from hydroxypropyl methyl cellulose acetate succinate L grade (HPMC-AS-L), hydroxypropyl methyl cellulose acetate succinate M grade (HPMC-AS-M), and hydroxypropyl methyl cellulose acetate succinate H grade (HPMC-AS-H).
10. The pharmaceutical compostion of embodiment 7, wherein the suspending agent is silicon dioxide.
11. The pharmaceutical composition of embodiment 7, wherein the carrier is lactose.
12. The pharmaceutical composition of embodiment 7, wherein the filler is lactose, microcrystalline cellulose, low substituted hydroxypropyl cellulose (L-HPC), or a combination thereof.
13. The pharmaceutical composition of embodiment 7, wherein the disintegrant is croscarmellose sodium.
14. The pharmaceutical composition of embodiment 7, wherein the glidant is silicon dioxide.
15. The pharmaceutical composition of embodiment 7, wherein the lubricant is sodium stearyl fumarate.
16. The pharmaceutical composition of embodiment 7, comprising:
   (i) an intragranular blend, wherein the intragranular blend comprises:
      (a) an amorphous spray granulate comprising:
         (i) the compound of Formula (I), wherein the compound is present in amorphous form, in an amount of 11.9 wt %;
         (ii) hydroxypropyl methyl cellulose acetate succinate in an amount of about 26.6 wt %;
      (b) silicon dioxide in an amount of about 1.3 wt %;
      (c) lactose in an amount of about 30.3 wt %;
   (ii) an extragranular blend, wherein the extragranular blend comprises:
      (d) lactose and microcrystalline cellulose in an amount of about 24.6 wt %;
      (e) croscarmellose sodium in an amount of about 2.8 wt %;
      (f) silicon dioxide in an amount of about 1.0 wt %; and
      (g) sodium stearyl fumarate in an amount of about 1.5 wt %.
17. The pharmaceutically acceptable composition of any one of embodiments 1-16, wherein the composition is in the form of a capsule or tablet.
18. A process of manufacturing a pharmaceutical composition according to embodiment 5 comprising:
   (i) preparation of an amorphous spray granulation, comprising:
      (a) suspending compound of Formula (I), a polymer and a suspending agent into an organic solution of acetone containing water;
      (b) mixing the suspension of (a) to form a dispersion of dissolved compound of Formula (I);
      (c) spraying the dispersion of (b) onto a carrier in a fluid bed dryer; to form an amorphous spray granulate;
   (ii) preparing an extragranular phase, wherein the extragranular phase comprises:
      (d) a filler;
      (e) a disintegrant;
      (f) a glidant; and
      (g) a lubricant
   (iii) blending the amorphous spray granulate (i) and the extrangranular phase (ii) to form a final blend.
19. The process of manufacturing a pharmaceutical composition according to embodiment 18, wherein the composition is filled into a capsule.
20. The process of manufacturing a pharmaceutical composition according to embodiment 18, wherein the composition is compacted into a tablet.
21. A method of treating a disease selected from the group consisting of gastrointestinal stromal tumors (GIST), NF-1-deficient gastrointestinal stromal tumors, succinate dehydrogenase (SDH)-deficient gastrointestinal stromal tumors, KIT driven gastrointestinal stromal tumors, PDGFRA driven gastrointestinal stromal tumors, melanoma, acute myeloid leukemia, germ cell tumors of the seminoma or dysgerminoma, mastocytosis, mast cell leukemia, lung adenocarcinoma, squamous cell lung cancer, glioblastoma, glioma, pediatric glioma, astrocytomas, sarcomas, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, lymphoblastic T-cell lymphoma, liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), myelodysplastic syndrome (MDS),and non-small cell lung cancer, comprising administering to a patient in need thereof a therapeutically effective amount of the composition of any one of embodiments 1-20.
22. The method of embodiment 21, wherein the disease is non-small cell lung cancer (NSCLC).
23. The use of the composition of any one of embodiments 1-20 for the preparation of a medicament for the treatment of a disease selected from the group consisting of gastrointestinal stromal tumors (GIST), NF-1-deficient gastrointestinal stromal tumors, succinate dehydrogenase (SDH)-deficient gastrointestinal stromal tumors, KIT driven gastrointestinal stromal tumors, PDGFRA driven gastrointestinal stromal tumors, melanoma, acute myeloid leukemia, germ cell tumors of the seminoma or dysgerminoma, mastocytosis, mast cell leukemia, lung adenocarcinoma, squamous cell lung cancer, glioblastoma, glioma, pediatric glioma, astrocytomas, sarcomas, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, lymphoblastic T-cell lymphoma, liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), myelodysplastic syndrome (MDS), and non-small cell lung cancer.
24. The use of embodiment 23, wherein the disease is non-small cell lung cancer (NSCLC).
25. A pharmaceutical composition of a compound represented by Formula (I)

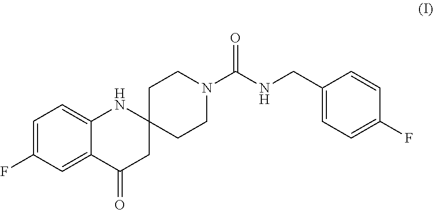 [SMILES: O=C1CC2(CCN(C(=O)NCc3ccc(F)cc3)CC2)Nc2ccc(F)cc21]

comprising:
(i) a nanosized crystalline spray granulate, comprising:
   (a) the compound of Formula (I), in crystalline Form A, wherein the crystals have been nano sized,
   (b) a polymer,
   (c) a surfactant;
   (d) a carrier;
(ii) an extragranular blend, wherein the extragranular blend comprises:
   (e) one or more fillers;
   (f) a disintegrant;
   (g) a glidant; and
   (h) a lubricant.
26. The pharmaceutical composition of embodiment 25 comprising:
   (i) a nanosized crystalline spray granulate, wherein the crystalline nanospray granulate comprises:
      (a) the compound of Formula (I), wherein the compound of Formula (I) is nanosized crystalline Form A in an amount of about 5 wt % to about 20 wt %,
      (b) a polymer in an amount of about 5 wt % to 20 wt %,
      (c) a surfactant in an amount of about 0.1 wt % to 1.0 wt %;
      (d) a carrier in an amount of about 20 wt % to 80 wt %;
   (ii) an extragranular blend, wherein the extragranular blend comprises:
      (e) one or more fillers in an amount of about 25 wt % to 50 wt %;
      (f) a disintegrant in an amount of about 2 wt % to 10 wt %;
      (g) a glidant in an amount of about 0.5 wt % to 2.0 wt %; and
      (h) a lubricant in an amount of about 0.5 wt % to 2.0 wt %.
27. The pharmaceutical composition of embodiment 26 comprising:
   (i) a nanosized crystalline spray granulate, wherein the crystalline nanospray granulate comprises:
      (a) the compound of Formula (I), wherein the compound of Formula (I) is nanosized crystalline Form A in an amount of 12.5 wt %,
      (b) a polymer in an amount of about 8.34%,
      (c) a surfactant in an amount of about 0.25 wt %;
      (d) a carrier in an amount of about 28.9 wt %;
   (ii) an extragranular blend, wherein the extragranular blend comprises:
      (e) one or more fillers in an amount of about 40 wt %;
      (f) a disintegrant in an amount of about 6 wt %;
      (g) a glidant in an amount of about 1.5 wt %; and
      (h) a lubricant in an amount of about 1.5 wt %.
28. The pharmaceutical composition of any embodiments 25-27, wherein the polymer is povidone or copovidone.
29. The pharmaceutical compostion of embodiment 28, wherein povidone is PVP K30.
30. The pharmaceutical composition of embodiment 27 comprising:
   (i) a crystalline nanospray granulate, wherein the crystalline nanospray granulate comprises:
      (a) the compound of Formula (I), wherein the compound of Formula (I) is nanosized crystalline Form A in an amount of 12.5 wt %,
      (b) povidone in an amount of about 8.34%,
      (c) sodium lauryl sulfate in an amount of about 0.25 wt %;
      (d) a lactose carrier in an amount of about 28.9 wt %:
   (ii) an extragranular blend, wherein the extragranular blend comprises:
      (e) lactose and microcrystalline cellulose in an amount of about 40 wt %;
      (f) croscarmellose sodium in an amount of about 6 wt %;
      (g) silicon dioxide in an amount of about 1.5 wt %; and
      (h) sodium steraryl fumarate in an amount of about 1.5 wt %.
31. A pharmaceutical compostion of any embodiments 25 to 30, wherein the crystals of the compound of Formula (I), crystalline Form A, have a median particle diameter (D50) of about 150 to 250 nm.
32. A process for preparing the pharmaceutical compostion according to any one of embodiments 25 to 31, said process comprising the steps of:
   (i) mixing a mixture comprising the compound of Formula (I), in crystalline Form A, wherein the crystals have been nano sized, a polymer, and a surfactant in a liquid medium, and
   (ii) adding said mixture to the carrier to form dry granulate.
33. The process according to embodiment 32, wherein step (i) is performed in a wet milling chamber.
34. The process according to embodiment 32 or 33, wherein the liquid medium is an aqueous solution.
35. The process according to embodiments 32 to 34, wherein the mixture of step (i) is dispersed onto the carrier and dried to form granulate.
36. The process according to any one of embodiments 32 to 35, wherein the process further comprises preparing the final dosage form by blending the granulate resulting from step (ii) with with extragranular phase, wherein the extragranular phase comprises one or more: filler; disintegrant; glidant; lubricant.
37. The process according to embodiment 36, wherein the final dosage form is encapsulated or tableted.
38. The process according to embodiment 37, wherein the final dosage form is tableted and the resulting tablet is further film coated.
39. A process for preparing a suspension comprising mixing the Compound of Formula (I), or a pharmaceutically acceptable salt thereof, or a free form thereof, at least one polymer, and optionally a surfactant, with a liquid medium.
40. The process according to embodiment 39, wherein the suspension is subjected to wet milling in order to reduce the size of crystals of Compound of Formula (I).
41. The suspension according to embodiment 40 wherein the median particle diameter (D50) of crystals of Compound of Formula (I) in said suspension is about 100 nm to 500 nm.
42. The suspension according to embodiment 41 wherein the median particle diameter (D50) of crystals of Compound of Formula (I) in said suspension is about 150 nm to 250 nm.
   Methods for makinq the pharmaceutical compositions

Compositions of the invention may suitably be made by combining the components as dry powders, for example tablets, capsules, or powder for reconstitution may be made by dry granulating the components of the tablet mix and optionally applying a film coating, for example a moisture barrier film, to the compressed tablet.

### Use of Compounds and Pharmaceutically Acceptable Compositions

As described generally above, Compound (1), and pharmaceutically acceptable solid compositions thereof described herein, are AKR1C3 inhibitors. The AKR1C3 inhibiting compounds of the present disclosure can, in some embodiments, find use in inhibiting the activity of AKR1C3.

In one aspect, the present disclosure provides methods for treating an AKR1C3 mediated disease or disorder in a subject in need thereof. In some embodiments, the method comprised administering tothe subject in need thereof a therapeutically effective amount of a pharmaceutical compositions disclosed herein, i.e., a pharmaceutical composition comprising Compound (1). In some emodiments, the disease or disorder is lung cancer, bladder cancer, cervical cancer, esphageal cancer, head and neck cancer, kidney cancer, and liver cancer. In some emodiments, lung cancer is non-small cell lung cancer (NSCLC), lung adenocarcinoma cancer, or lung squamous cell cancer. In some emobdiments, the administration is oral administration.

In another aspect, the present disclosure provides a pharmaceutical compositions as disclosed herein, i.e. a pharmaceutical composition comprising Compound (I), for use in treating an AKR1C3 mediated disease or disorder in a subject in need thereof. In yet another aspect, the present disclosure provides a pharmaceutical compositions as disclosed herein, i.e. a pharmaceutical composition comprising Compound (I), for the manufacture of a medicament for treating an AKR1C3 mediated disease or disorder in a subject in need thereof. In some emodiments, the disease or disorder is lung cancer, bladder cancer, cervical cancer, esphageal cancer, head and neck cancer, kidney cancer, and liver cancer. In some emodiments, lung cancer is non-small cell lung cancer (NSCLC), lung adenocarcinoma cancer, or lung squamous cell cancer.

The term “treatment” is used interchangeably herein with the term “therapeutic method” and refers to both 1) therapeutic treatments or measures that cure, slow down, lessen symptoms of, and/or halt progression of a diagnosed pathologic condition, disease or disorder, and 2) and prophylactic/preventative measures. Those in need of treatment may include individuals already having a particular medical disease or disorder, as well as those who my ultimately aquire the disorder (i.e., those at risk or needing preventative measures).

The term “subject” as used herein refers to any individual or patient to which the subject methods are preformed. Generally, the subject is human, although as will be appreciated by those in the art, the subject may be an animal.

The terms “therapeutically effective amount”, “effective dose”, “therapeutically effective dose”, “effective amount”, or the like refer to the amount of a subject compound that will elice the biological or medical response in a tissue, system, animal or human that is being south by adminsitering said compound.Generally, the response is either amelioration of symptoms in a patient or a dsired biological outcome. In some embodiments, such amound should be sufficient to inhibit AKR1C3.

In some embodiments, an effective amount of a AKR1C3 inhibiting compound of the invention is an amount that ranges from about 10 mg to 1000 mg. In further embodiments the amount ranges from about 10 mg to about 50 mg, from about 50 mg to about 100m g, from about 100 mg to 200 mg, from about 200 mg to 300 mg, from about 300 mg to 400 mg, from about 400 mg to 500 mg, from about 500 mg to 1000 mg. The amount can be a single dose amount or can be a daily amount. In some embodiments, an effection amount of AKR1C3 inhibiting compound is about 100 mg.

### Definitions

As used herein, the term “about”, when used in reference to an amount refers to the stated value ±10% of said value. In some embodiments, “about” refers to the stated value±5% of said value, 2% of said value, or+1% of said value.

As used herein, the terms “administer,” “administering,” and “administration,” refer to any method which, in sound medical practice, delivers a provided composition, or an active agentcontained therein, to a subject in such a manner as to provide a therapeutic effect.

As used herein, the phrases an “effective amount” or a “therapeutically effective amount” of an active agent or ingredient, or pharmaceutically active agent or ingredient, refer to an amount of the pharmaceutically active agent sufficient enough to have a therapeutic effect upon administration. Effective amounts of the pharmaceutically active agent will vary with the kind ofpharmaceutically active agent chosen, the particular condition or conditions being treated, the severity of the condition, the duration of the treatment, the specific components of the composition being used, and like factors. Generally, the response is either amelioration of symptoms in a patient or a desired biological outcome. In some embodiments, such amount should be sufficient to inhibit a c-kit kinase and treat a c-kit kinase related disease or disorder.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Such suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

As used herein the term “preservative” refers to any known pharmaceutically acceptable preservative that functions by inhibiting bacteria, fungi, yeast, mold, other microbe, and/or by inhibiting oxidation. Suitable preservatives include but are not limited to antimicrobial agents and/or antioxidants. Suitable antimicrobial agents can include but are not limited to benzoates, benzyl alcohol, sodium benzoate, sorbates, propionates, and nitrites. Suitable antioxidants can include but are not limited to vitamin C, butylated hydroxytoluene (BHT), sulphites, and vitamin E. Other such preservatives for use in the present invention are described above and herein.

The term “prevent,” “preventing,” or “prevention,” as used herein refers to any reduction, no matter how slight, of a subject's predisposition or risk for developing a condition,disease, disorder or symptom thereof. For purposes of prevention, the subject is any subject, andpreferably is a subject that is at risk for, or is predisposed to, developing a condition, disease, disorder. The term “prevention” includes either preventing the onset of a clinically evidentcondition, disease, disorder altogether or preventing the onset of a pre-clinically evident condition, disease, disorder in individuals at risk. This includes prophylactic treatment of subjects at risk ofdeveloping condition, disease, disorder.

As used herein, the term “solvent” refers to any pharmaceutically acceptable mediumwhich is a liquid at ambient temperature, in which one or more solutes can be dissolved, or one or more substances can be partially dissolved or suspended. Numerous solvents are well known in the chemical and pharmaceutical arts and are contemplated herein and below.

The phrase “substantially pure” as used herein refers to an individual compound form, which is substantially devoid of all other fo1 ms, as well as degradation products of a form, and any residual solvent, and is at least 85% pure on a % weight basis, unless otherwise specified. The compound form can have at least 90% purity on a % weight basis, at least 93% purity on a % weight basis, at least 95% purity on a % weight basis, or at least 97%, 98%, 99%, or 99.5% purityon a % weight basis.

As used herein, “subject” or “individual” or “animal” or “patient” or “mammal,” refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder,or condition is totally cured. A useful composition herein needs only to reduce the severity of a disease, disorder, or condition, reduce the severity of symptoms associated therewith, provide improvement to a patient or subject's quality of life, or delay or inhibit the onset of a disease, disorder, or condition.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and thatthere are processes and methods according to the present invention that consist essentially of, or consist of, the recited processing steps.

As used herein, all percentages are by weight of the total composition (i.e., wt %), unless otherwise specified.

Any concentration ranges, percentage range, or ratio range recited herein are to be understood as expressly disclosing and including any concentrations, percentages or ratios of anyinteger within that range and fractions thereof, such as one tenth and one hundredth of an integer, and any sub-range falling within a range, unless otherwise indicated.

Any number range recited herein relating to any physical feature, including for example, polymer subunits, size or thickness, are to be understood as expressly disclosing and including any integer or fraction of an integer within a disclosed range, or any sub-range within a disclosed range, unless otherwise indicated.

For the purpose of clarity, any element or feature of any method or composition or process described herein, can be combined with any other element or feature of any other methodor composition or process described herein. Other terms as used herein are meant to be defined by their well-known meanings in theart.

All features of each of the aspects of the disclosure apply to all other aspects mutatis mutandis. Each of the references referred to herein, including but not limited to patents, patent applications and journal articles, is incorporated by reference herein as though fully set forthin its entirety,

In order that the disclosure described herein may be more fully understood, the following examples are set forth. It should be understood that these examples are for illustration purposes only and are not to be construed as limiting this disclosure in any matter.

### EXAMPLES

### Example 1

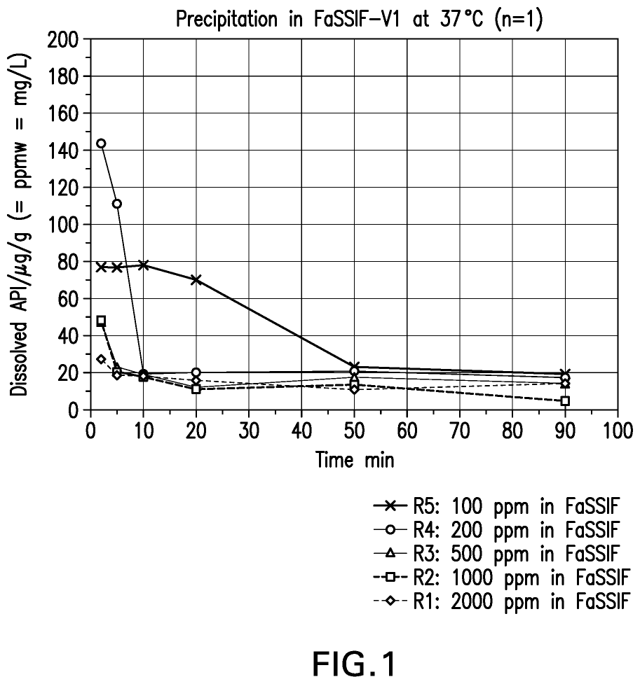

The supersaturation potential of Compound (1) was studied by mixing drug dissolved in 1 mL DMSO into 99 mL FaSSIF-V1 stirred at 37° C. (FIG. 1). At higher drug loads of 500, 1000 or 2000 ppmw (or pg/g or pg/mL) Compound (1) precipitated practically instantaneously and attained a constant steady state concentration of approx. 20 μg/mL or below within 5 minutes.. At the 90 min final sampling the solids were confirmed to be crystalline Compound (1) by XRPD. At lower drug loads, supersaturation was maintained longer, e.g. approx. 110 μg/mL drug dissolved at 5 min at 200 ppmw drug load and approx. 70 μg/mL drug dissolved till 20 min at 100 ppmw drug load. Situation after desupersaturation was similar as described for higher drug loads. Overall, Compound (1) appears to be a rather poorly supersaturating compound as it is very fast recrystallizing into free form Mod.A. Only low supersaturation can be maintained for relatively short time.

### Example 2

The following example illustrates the initial feasibility assessment of an amorphous solid dispersion formulation. An amorphous solid dispersion (ASD) was initially evaluated as a potential formulation for Compound (1). Solid dispersion screening was performed by lyophilization of solid Compound (1) with a polymer. Miscibility trials of Compound (1) were performed with Kollidon® VA64, HPMC-AS-LF, Eudragit® L100-55, and HPMC 603, by conducting a DSC measurement of the lyophilized material to determine if the material was amorphous or crystalline. As shown in TABLE 2, HPMC 603 with 40% drug loading recrystallized after 3 weeks under stressed conditions. Kollidon® VA64 at both 50% and 40% drug loading recrystallized after 5 weeks. HPMC-AS-LF and Eudragit® L100-55 remained amorphous under stressed conditions. However, HPMC-AS-LF and Eudragit® L100-55 both have significant impurities after 5 weeks until stressed conditions, according to UPLC analysis.

TABLE 2
Initial Stability Evaluation of Compound
(I) in Solid Dispersion Polymers

| | Drug- | | | | |
|---|---|---|---|---|---|
| | load | 3 weeks 50° C./75% RH | | 5 weeks 50° C./75% RH | |
| Polymer | (%) | mDSC | UPLC (%) | mDSC | UPLC (%) |
| Kollidon ® | 40 | Amorphous | 98.85 | Crystalline | 98.86 |
| VA 64 | | | | | |
| Kollidon ® | 50 | Crystalline | 98.87 | Crystalline | 98.89 |
| VA 64 | | | | | |
| HPMC- | 40 | Amorphous | 97.58 | Amorphous | 96.44 |
| AS-LF | | | | | |
| Eudragit ® | 50 | Amorphous | 97.80 | Amorphous | 97.30 |
| L100-55 | | | | | |
| HPMC 603 | 40 | Crystalline | 98.91 | Crystalline | 99.02 |
| HPMC 603 | 30 | Amorphous | 98.88 | Amorphous | 98.98 |
| 100% Drug | 100 | N/A | 99.09 | N/A | 99.05 |
| Load | | | | | |

Initial purity of Compound (I) was 99.3%

Based on the miscibility, stability (5 week stabililty at 50° C./75% RH by mDSC), kinetic solubility data (Example 1), and poor supersaturation and very fast recrystallization of the compound, viability of an ASD formulation for Compound (1) was considered to be very challenging. Consequently, a first option was to formulate Compound (1) as crystalline form, but enhancing bioavailability through nanosizing the drug crystals. It was determined that the second formulation option via amorphous solid dispersion would be very challenging and only beneficial when the drug can be protected in amorphous form until reaching the site of absorption (duodenum, intestine) and released gradulallly to maintain supersaturation low enough to prevent fast recrystallization.

### Example 3: Beagle Dog Studies

The following example illustrates three PK beagle dog studies that were conducted using various formulations containing Compound (1) at a dose of 100 mg/animal. AUC, Cmax, and Tmax data from the three studies are summarized in TABLE 3. The first dog PK Study (Dog Study #1) was done to compare spray dried granules of micronized crystalline drug (MSG), and spray dried granules of nanosized crystalline drug (NSG), against an HPMC-AS based hot-melt extruded amorphous solid dispersion (HME-ASD). All powders were dosed in a hard gelatin capsule (HGC). The HME-ASD formulation performed better than MSG and NSG.

The second dog PK study (Dog Study #2) compared nanosized crystalline granules (NSG) finished to film coated tablets (FCT), with spray dried amorphous solid dispersion (ASD) made with neutral polymers (i.e. HPMC or Soluplus®). The PK parameters were comparable between all formulations.

The third dog PK study (Dog Study #3) compared exposures of HPMC (neutral polymer) based amorphous spray granules (ASG) with and without an enteric polymer coating of HPMC-AS-L or HPMC-AS-H. The PK parameters were comparable between all formulations.

Based on the results from the three dog PK studies, summarized in TABLE 3, it was concluded that only an amorphous solid dispersion (ASD) with an enteric polymer, e.g. HPMC-AS, would provide maximum exposure. As the exposures from the ASDs with neutral polymers were similar to the nanosized crystalline drug formulations, the approach of spray dried granules of nanosized crystalline drug (NSG) was deemed another attractive formulation opportunity, particlularly considering the substantial exposure advantage of NSG over poor exposure from the spray dried granules of micronized crystalline drug (MSG).

TABLE 3
Beagle Dog PK studies

| | Dog Study #1 | | | | | |
|---|---|---|---|---|---|---|
| | | | HPMC-AS | Dog Study #2 | | |
| | Micronized | Nanosized | based | NSG | | |
| | Crystalline | Crystalline | amorphous | finished | HPMC | Soluplus ® |
| | Spray | Spray | solid | to film | based | based |
| Formulation | Granules | Granules | dispersion | coated | ASD | ASD |
| Type | (MSG) | (NSG) | (HME-ASD) | tablets | (neutral) | (neutral) |
| AUC [nM*h] | 2680 | 11700 | 25000 | 12700 | 14100 | 10100 |
| (Std. Dev.) | (1250) | (1370) | (9000) | (3790) | (1470) | (5340) |
| Cₘₐₓ [nM] | 792 | 3570 | 8850 | 3420 | 2800 | 2570 |
| (Std. Dev.) | (458) | (1150) | (4110) | (1550) | (817) | (1350) |
| Tₘₐₓ [h] | 1.5 | 1.67 | 1.5 | 1.0 | 2.0 | 2.0 |
| (Std. Dev.) | (0.87) | (0.58) | (0.87) | (0.5-4.0) | (2.0-4.0) | (2.0-2.0) |

| | Dog Study #3 | | |
|---|---|---|---|
| | HPMC based | Neutral | Neutral |
| | ASD spray | ASG coated | ASG coated |
| | granules | with | with |
| Formulation | (neutral | enteric | enteric |
| Type | ASG) | HPMC-AS-L | HPMC-AS-H |
| AUC [nM*h] | 14300 | 16000 | 14900 |
| (Std. Dev.) | (5690) | (5030) | (11500) |
| Cₘₐₓ [nM] | 2610 | 3240 | 1860 |
| (Std. Dev.) | (637) | (1088) | (178.3) |
| Tₘₐₓ [h] | 2.0 | 2.0 | 0.75 |
| (Std. Dev.) | (1.06-2.0) | (2.0-2.0) | (0.5-2.0) |

### Example 4: Nanosuspension spray granulate and Hot Melt Extrusion

The following Example illustrates a nanosuspension spray granulate (NSG). Unmilled Compound (1) has a median particle size distribution (D50) between approximately 50 and 200 μm. Compound (1) was micronized by jet milling to obtain a very fine drug substance with a D50 between approximately 1 and 3 μm. A nanosuspension was prepared by mixing 10% of the jet milled Compound (1), 2% PVP K30, and 0.1% SLS to water. A nanosuspension spray granulate (NSG) was then prepared by spray drying the nanosuspension and additional PVP K30 and SLS onto a sugar core carrier. The NSG was then blended with additional extragranular excipients, e.g. mannitol, lactose, Avicel PH012 (MCC), Croscarmellose sodium, Crospovidone, silicon dioxide, sodium steraryl fumarate. The blend was compressed into tablets using a tablet press and film coated.

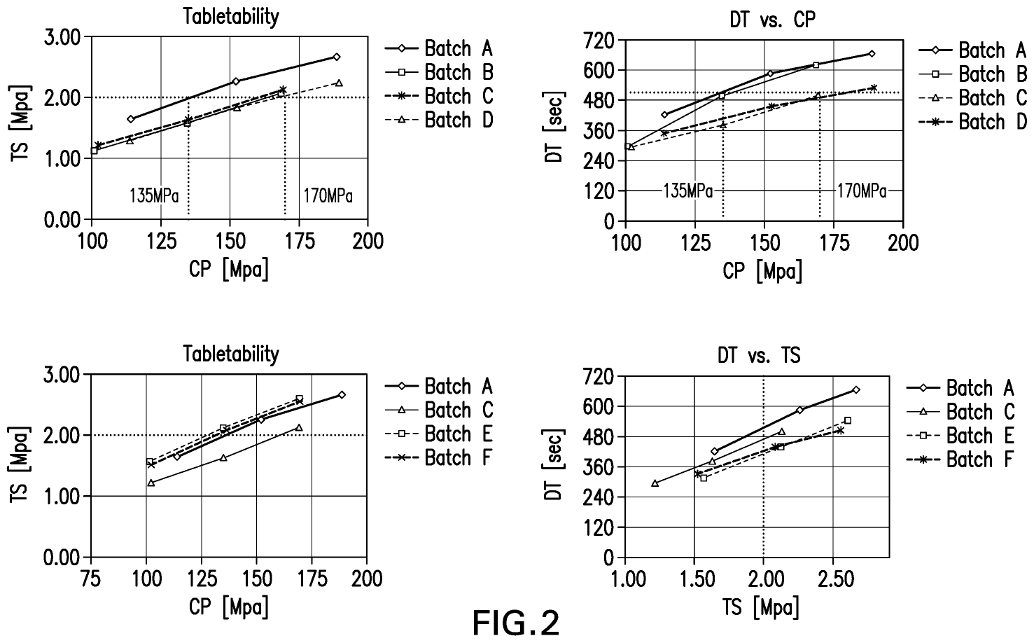

Several batches of NSG tablet were produced to evaluate the best composition. Lactose SD, Mannitol SD were used as sugar cores in NSG. Lactose SD, Mannitol SD, and Microcrystalline Cellulose (MCC) were used as a fillers and croscarmellose sodium and crospovidone were evaluated as disintegrants to produce the final tablets. Table 4 details the excipients used in several batches to evaluate these excipients' impact on compaction properties. FIG. 2 illustrates the impact of the excipients on compaction properties.

Formulations were optimized to target a disintegration time under 10 minutes at 2 MPa. The ratio between soluble and insoluble filler was optimized to 40% soluble filler and 60% insoluble filler.

TABLE 4
Excipient Evaluation on Compaction Properties

| | | Batch | | Batch | | Batch | | Batch | | Batch | | Batch | |
|---|---|---|---|---|---|---|---|---|---|---|---|---|---|
| Ingredient | Function | A | | B | | C | | D | | E | | F | |
| Compound I | Active | 50.12 | | 44.85 | | 47.34 | | 53.25 | | 47.58 | | 46.87 | |
| NSG | | | | | | | | | | | | | |
| Mannitol SD | Filler | 13.63 | | 29.10 | | 23.66 | | 22.75 | | — | | 17.65 | |
| Lactose SD | Filler | — | | — | | — | | — | | 17.37 | | — | |
| MCC102 | Filler | 27.25 | | 14.55 | | 20.00 | | 15.00 | | 26.05 | | 26.48 | |
| Croscarmellose | Disin- | 6.00 | | — | | 6.00 | | 6.00 | | 6.00 | | 6.00 | |
| Sodium | tegrant | | | | | | | | | | | | |
| Crospovidone | Disin- | — | | 8.00 | | — | | — | | — | | — | |
| | tegrant | | | | | | | | | | | | |
| Sodium Steryl | Lubricant | 1.50 | | 2.00 | | 1.50 | | 1.50 | | 1.50 | | 1.50 | |
| Fumarate | | | | | | | | | | | | | |
| Silicon Dioxide | Glidant | 1.50 | | 1.50 | | 1.50 | | 1.50 | | 1.50 | | 1.50 | |
| Total | | 100.00 | | 100.00 | | 100.00 | | 100.00 | | 100.00 | | 100.00 | |
| Tablet Weight | | 680 | mg | 760 | mg | 720 | mg | 640 | mg | 720 | mg | 720 | mg |
| Punch Size | | 17 | mm | 18 | mm | 18 | mm | 17 | mm | 18 | mm | 18 | mm |

The final composition of the NSG can be found in Table 5.

TABLE 5
Final composition of NSG

| | | |
|---|---|---|
| Ingredient | Function | wt % |

Nanosuspension Spray Granulate (NSG)

| | | |
|---|---|---|
| Compound I | Active | 25.0 |
| PVP-30 | Polymer | 16.67 |
| Sodium Lauryl Sulfate (SLS) | Surfactant | 0.5 |
| Lactose SD | Sugar core | 57.8 |

NSG + Extragranular blend

| | | |
|---|---|---|
| Nanosupsension Granulate | Active | 50.0 |
| Lactose Monohydrate SD | Filler | 16.4 |
| Microcrystalline Cellulose | Filler | 24.6 |
| Croscarmellose sodium | Disintegrant | 6.0 |
| Sodium Stearyl fumurate | Lubricant | 1.5 |
| Colloidal Silicon Dioxide | Glidant | 1.5 |

Manufacturing Process of Nanosuspension Spray Granulate tablet:
a. Dissolve the binder, e.g. polyvinylpyrrolidone (PVP), into water under stirring.
b. Add surfactant, e.g. sodium lauryl sulfate (SLS), to the solution of step a and dissolve under stirring.
c. Add Compound (1) to the solution of step b and suspend under stirring.
d. Perform milling, e.g. wet-ball milling, with the suspension of step c.
e. Dissolve required amounts of SLS and polyvinylpyrrolidone in the additional purified water under stirring.
f. Weigh required amount step d suspension and add to the solution of step e to complete the suspension for spraying, e.g. spray granulation.
g. Load the inert substrate (carrier particle), e.g. lactose SD or mannitol SD.
h. Perform spraying, e.g. spray granulation, by spraying the suspension from step e to the inert substrate, e.g. Lactose SD or mannitol SD200, from step g.
i. The granule particles from step h were blended with and extragranular blend of lactose, microcrystalline cellulose, croscarmellose sodium, silica, and sodium steraryl fumarate.
j. The blend mixture from step i was introduced in a capsule or compressed to form a tablet.

A holt melt extrusion (HME) approach was also attempted. Although amorphous solid dispersion (ASD) of Compound (1) and differnt polymers could be prepared, this approach was quickly abandoned due to thermal degradation of Compound (1), since it was necessary to extrude the compound-polymer mixture at temperatures in excess of 160° C.

### Example 5: Amorphous Solid Dispersion Spray Granulate Development

The following Example illustrates the chemical and physical stability of Compound (1) in various polymers when stored over time at elevated temperatures and varying relative humidity conditions. Amorphous Solid Dispersion (ASD) powders were prepared by spray drying mixtures of Compound (1) and various polymers from organic solution.

All powders exhibited good chemical and physical stability when stored ambient, e.g. for at least 3 months. However, storage at elevated temperature revealed significant differences, refer to Table 6. Generally, impurities were increasing with temperature, moisture and time. The highest impurity levels were observed with enteric polymers. HPMC-AS had greater impurities than Eudragit L100-55. This may point towards particular drug degradation in presence of acidic functional groups, in line with earlier described drug instability in acidic solutions. Lower but still significant impurity levels were observed with neutral HPMC. In addition, more drug and less polymer seems to reduce the total impurities generated. Interestingly, very little degradation occurred with neutral polymer Soluplus®. On the other hand, Soluplus® was the only polymer where Compound (1) recrystallized already at 40° C. (8 weeks, 75% RH), more pronounced recrystallization at 60° C. Another unfavorable property of the Soluplus® materials was softening at 40° C., 75% RH or 60° C., 50% RH leading to conversion of the powder into a hard, sintered plug. Powder sintering and plug formation was also observed for Eudragit® L100-55 after storage at 40° C., 75% RH or 60° C., 11% RH, however no recrystallization was noted. HPMC-AS powders consolidated rather slightly and the plug could be redispersed, no recrystallization was indicated. All HPMC powders remained flowable with no signs of recrystallization. TGA results indicated relatively limited moisture absorption, depending on storage condition, with the lowest levels associated to HPMC-AS as ASD polymer.

HPMC based ASG

The following development wave translated ASD manufacture via spray drying to spray granulation with the opportunity to cover the granules with an enteric coating. To harvest previous chemical and physical stability experience, HPMC was used as polymer for ASD or Amorphous Spray Granules (ASG). Other ASG ingredients were Mannitol SD as carrier and small amounts of silica. The drug and polymer were dissolved in acetone/water for spray solution preparation. By further spray granulation, the neat ASG was added with an insulation layer of HPMC, silica, SLS and on top with enteric layers of HPMC-AS-L or HPMC-AS-H. All three types of granules were tested for bioperformance in a third dog PK study #3 (as discussed above). Exposures were not much different between the HPMC based ASGs, no matter whether only carrying the neutral seal layer or covered with additional enteric layer of HPMC-AS-L or HPMC-AS-H. Furthermore, only the neat HPMC based ASG was exhibiting little drug degradation, i.e. total impurities of about 0.1% after 8 weeks at 40° C., 75% RH, while it was significant with enteric coating, e.g. with total impurities of about 1.5% and 0.9%, respectively, after 8 weeks at 40° C., 75% RH. In conclusion, addition of a neutral seal layer did not protect the drug from more degradation in conjunction with HPMC-AS nor did the layering help to improve bioperformance beyond the levels provided already by the nano crystal approach.

HPMC-AS based ASG

The final approach was to go back to an enteric polymer, i.e. HPMC-AS based ASD, as chemical and physical stability seemed acceptable at ambient storage. The manufacturing approach was similar to the foregoing. However, the carrier was changed to Lactose SD. The spray solution contained about 9% solids (Compound (I), HPMC-AS-LF, silica) dissolved/suspended in acetone/water 9/1 (w/w). In line with expectation, physical and chemical stability of the HPMC-AS based ASG was uncritical with regard to ambient storage at 8 weeks or 4 months. Slightly increased stress storage at 30° C., 75% RH indicated first drug degradation after 8 weeks. On the other end, at 60° C., 11% RH significant drug degradation was observed. To support shelf life, it was decided to qualify degradation levels of about 2%.

Another liability of the ASG was the residual acetone content of 1.8-2.3%, to some extent also reflected in more mass loss by TGA from HPMC-AS based ASG. By applying vacuum drying (approx. 20 mbar) at 50 or 60° C. for about 1 day, residual acetone could be reduced to levels below 0.5% without notable degradation.

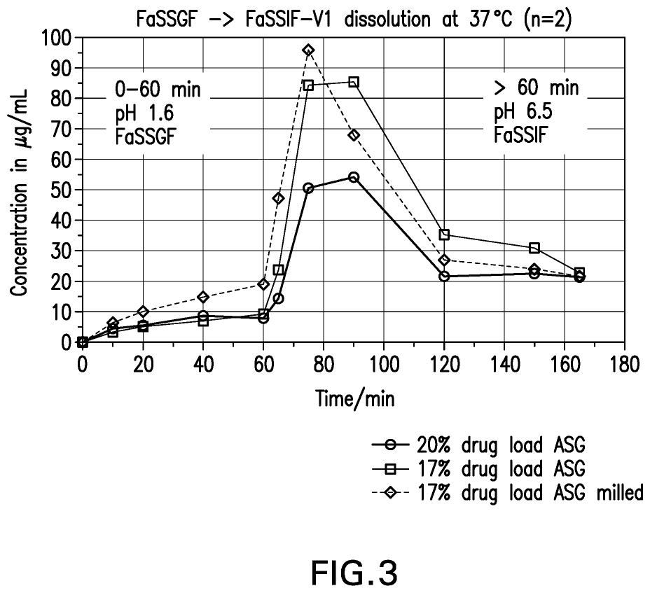

Powder properties of the ASG batches were favorable, i.e. high bulk density, good flowability, reasonable PSD. Nevertheless, milling had to be explored for different purposes. At first hand hammer milling was performed in order to tailor the granule size towards better compactibility. Two major limitations were encountered: First, when hammer milling bigger amounts material built up on the sieve, e.g. observed with 0.2 mm mesh. Second, size reduction, e.g. from median particle size (D50) of 229 μm to D50 of 155 μm, led to a more spiked drug release with shorter supersaturation, as can be taken from FIG. 3. This is considered somewhat undesirable as the target is to maximize the drug concentration in solution for as long as possible. FIG. 3 shows at the same time that also drug load increase to 20% in ASG (35% in the ASD layer) is detrimental to that. On the other hand and independent of the observed in-vitro dissolution, pin milling had to be applied to reduce the granules to finer powders with the aim of improving further the compactibility or matching the oral gavage orifice for dosing rodents. For example, applying pin milling at different impact on ASG yielded powders with median particle size (D50) between approximately 20 and 100 μm.

TABLE 6
Chemical and Physical Stability of Compound (I)
in various polymers under various conditions

| Batch # | | | | | | | | |
|---|---|---|---|---|---|---|---|---|
| 1 | | | 2 | | | 3 | | |
| Variant | | | | | | | | |
| 20% Compound (I), | | | 30% Compound (I), | | | 40% Compound (I), | | |
| 80% HPMC | | | 70% HPMC | | | 60% HPMC | | |
| Polymer | | | | | | | | |
| Neutral | | | Neutral | | | Neutral | | |
| Conditions | | | | | | | | |
| 40°, | 40°, | 60°, | 40°, | 40°, | 60°, | 40°, | 40°, | 60°, |
| 11% | 75% | 50% | 11% | 75% | 50% | 11% | 75% | 50% |
| RH | RH | RH | RH | RH | RH | RH | RH | RH |

Storage Time (% Assay/% Impurities/TGA wt. %, XRPD)

| | | | | | | | | | |
|---|---|---|---|---|---|---|---|---|---|
| 4 weeks | 102.7 | 98.2 | 100.3 | 101.2 | 96.8 | 96.7 | 101.3 | 97.6 | 99.9 |
| | <0.1 | <0.1 | 0.59 | <0.1 | 0.23 | 0.54 | <0.1 | 0.14 | 0.26 |
| | 2.0 am. | 3.8 am. | 3.0 am. | 2.3 am. | 4.7 am. | 3.3 am. | 3.0 am. | 5.3 am. | 3.6 am. |
| 8 weeks | 101.5 | 98.4 | 99.1 | 100.1 | 97.4 | 98.3 | 100.7 | 98.3 | 99.5 |
| | 0.11 | 0.63 | 1.06 | 0.11 | 0.47 | 1.03 | 0.11 | 0.32 | 0.58 |
| | 3.8 am. | 4.8 am. | 3.6 am. | 2.9 am. | 4.1 am. | 2.3 am. | 2.7 am. | 3.6 am. | 2.9 am. |

| Batch # | | |
|---|---|---|
| 4 | 5 | 6 |
| Variant | | |
| 30% Compound (I), | 30% Compound (I), | 30% Compound (I), |
| 70% Soluplus ® | 70% HPMC-AS-LF | 70% Eudragit ® L100-55 |
| Polymer | | |
| Neutral | Enteric | Enteric |

Conditions

| | | | | | | |
|---|---|---|---|---|---|---|
| 40°, | 40°, | 60°, | 40°, | 60°, | 40°, | 60°, |
| 11% | 75% | 50% | 75% | 50% | 75% | 50% |
| RH | RH | RH | RH | RH | RH | RH |

Storage Time (% Assay/% Impurities/TGA wt. %, XRPD)

| | | | | | | | |
|---|---|---|---|---|---|---|---|
| 4 weeks | 99.9 | 96.1 | 98.7 | 99.6 | 99.1 | 99.0 | 100.1 |
| | <0.1 | <0.1 | <0.1 | 0.39 | 1.75 | 0.2 | 0.96 |
| | 2.3 am. | 5.1 am. | 3.0 trace | 1.8 am. | 1.0 am. | 3.1 am. | 1.1 am. |
| 8 weeks | 99.9 | 96.5 | 94 | 97.6 | 96.5 | 97.5 | 97.5 |
| | 0.11 | 0.11 | 0.23 | 1.06 | 4.50 | 0.51 | 2.07 |
| | 1.5 am. | 4.6 trace | 3.5 cryst. | 2.4 am. | 1.2 am. | 3.0 am. | 1.1 am. |

### Example 6: PK performance of HPMC-AS ASG in Cynomolqus monkeys

The following example illustrates the bioperformance of the HPMC-AS based ASG in cynomolgus monkeys. The (hammer milled) granules were suspended in 50 mM NaH2PO4 buffer (pH 4.6) and administered via oral gavage. Rather linear exposure (AUC) increase has been found at doses from 3 to 15 mg/kg while it was under proportional at 50 mg/kg, see Table 7. The latter may have been confounded to some extend by somewhat incompletely dosing 2 out of 3 animals. However, higher driving force for Compound (1) desupersaturation at high dose could be a key factor.

TABLE 7

| | Dose/ | Tmax/ | Cmax/D/ | AUC0-24 h/D/ |
|---|---|---|---|---|
| Formulation | mg/kg | h | nM*kg/mg | nM*hr*kg/mg |
| HPMC-AS-L | 3 | 1.0 [1.0-4.0] | 3920 ± 306 | 15800 ± 9370 |
| based ASD | 15 | 1.0 [0.5-4.0] | 8340 ± 5600 | 19500 ± 4300 |
| granules | 50 | 1.0 [0.5-2.0] | 1320 ± 2120 | 4060 ± 6280 |
| (ASG hammer | | | | |
| milled), | | | | |

### Example 7: PK performance of HPMC-AS ASG formulation in Wistar Rats

The following example illustrates the bioperformance of the HPMC-AS based ASG and served to check dose dependent exposure of the final HPMC-AS based ASG. Pin milled HPMC-AS based ASG was suspended in 50 mM NaH₂PO₄ buffer (pH 4.6) and administered via oral gavage. At 100 mg/kg dose the bioperformance of the HPMC-AS based ASG was similar to the one of the nanosuspension. However, significant outperformance of the HPMC-AS based ASG resulted at 600 mg/kg when compared to limited exposure increase of the nanosuspension at 300 or 1000 mg/kg dose, as shown in Table 8.

TABLE 8

| | Dose | | | Cmax/D | | | AUC0-24/D |
|---|---|---|---|---|---|---|---|
| | Level | | Cmax | [(ng/mL)/ | Tmax | AUC0-24 | [(h*ng/mL)/ |
| Study | (mg/kg/day) | Sex | (ng/mL) | Mg/kg/day)] | (h) | (h*ng/mL) | (mg/kg/day)] |
| Nano- | 100 | M | 3990 | 39.9 | 1 | 31100 | 311 |
| suspen. | | F | 12900 | 129 | 1 | 82400 | 824 |
| | 300 | M | 5720 | 19.1 | 1 | 32500 | 108 |
| | | F | 15500 | 51.5 | 1 | 99800 | 333 |
| | 1000 | M | 9100 | 9.1 | 1 | 97500 | 98 |
| | | F | 15900 | 15.9 | 3 | 175000 | 175 |
| ASG | 100 | M | 7850 | 78.5 | 0.7 | 36722 | 367 |
| | | F | 21700 | 217 | 1.3 | 83691 | 837 |
| | 600 | M | 18000 | 30 | 0.5 | 217410 | 362 |
| | | F | 37500 | 62.5 | 0.5 | 258803 | 431 |

### Example 8: Manufacturinq Process of Amorphous Solid Dispersion Spray Granulate and Capsule Formulation

The following example describes the manufacturing process for the amorphous spray granule formulation of Compound (1).

Part A. Granules (ASG):
1. Combine Compound (1), silicon dioxide and HPMC-AS into a solution of acetone and water.
2. Mix in a suitable vessel until a visually homogenous, fine yellow dispersion is formed
3. Sieve Lactose Monohydrate SD.
4. Load fluid bed granulator with Lactose Monohydrate SD from step 3 and perform fluid bed granulation with the dispersion from step 2
5. Mill the fluid bed granules from step 4

Part B. Hard Gelatin Capsules (HGC):
1. Add the materials into a suitable container: Lactose monohydrate SD, Compound (1) granules (from Part A, step 5), silicon dioxide, croscarmellose sodium, sodium steraryl fumarate, microcrystalline cellulose and blend
2. Sieve the blend from step 1
3. Blend the mixture from step 2
4. Encapsulate the final blend from step 3

### Example 9: Manufacturinq Process of Amorphous Solid Dispersion Spray Granulate and Tablet Formulation

The following example describes the manufacturing process for the amorphous spray granule formulation of Compound (1).

Part A. Granules (ASG):
1. Combine Compound (1), silicon dioxide and HPMC-AS into a solution of acetone and water.
2. Mix in a suitable vessel until a visually homogenous, fine yellow dispersion is formed
3. Sieve Lactose Monohydrate SD.
4. Load fluid bed granulator with Lactose Monohydrate SD from step 3 and perform fluid bed granulation with the dispersion from step 2
5. Mill the fluid bed granules from step 4

Part B. Film Coated Tablet (FCT)
1. Add the materials into a suitable container: Compound (1) granules (from Part A, step 5), silicon dioxide, croscarmellose sodium, sodium steraryl fumarate, microcrystalline cellulose and blend
2. Sieve the blend from step 1
3. Blend the mixture from step 2
4. Compress the final blend from step 3 to tablets
5. Film coat the tablets from step 4

Part C. Film Coated Tablet (FCT)
1. Add the materials into a suitable container: Compound (1) granules (from Part A, step 5), silicon dioxide, low substituted hydroxypropyl cellulose (L-HPC), sodium steraryl fumarate and blend
2. Sieve the blend from step 1
3. Blend the mixture from step 2
4. Compress the final blend from step 3 to tablets
5. Film coat the tablets from step 4

1. A pharmaceutical composition of a compound represented by Formula (I) 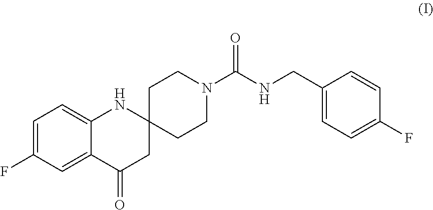 [SMILES: O=C1CC2(CCN(C(=O)NCc3ccc(F)cc3)CC2)Nc2ccc(F)cc21]
Comprising the Compound of Formula (I) stabilized in amorphous form with a polymer.

2. The pharmaceutical composition of claim 1, wherein the Compound of Formula (I) is present in about 5 to 80 wt %, about 10 to 50 wt %, about 25 to 40 wt %, or about 30 wt %.

3. The pharmaceutical composition of claim 1, wherein the polymer is selected from: hydroxypropyl methylcellulose, Hydroxypropyl methylcellulose acetate succinate (HPMC-AS), hydroxypropyl methylcellulose phtalate, hydroxypropyl cellulose, povidone (PVP), copovidone (PVP VA 64), cellulose acetate, cellulose acetate phtalate, or polyacrylates, e.g. ammonio methacrylate copolymers (e.g. Eudragit RS/RL), METHACRYLIC ACID-ETHYL ACRYLATE COPOLYMER (e.g. Eudragit L100 or L100-55), polyvinylacetate, polyvinylacetate phtalate, polyvinyl caprolactam-polyvinyl acetate-polyethylene glycol graft copolymer (Soluplus®).

4. The pharmaceutical composition of claim 3, wherein the polymer is HPMC-AS-L, HPMC-AS-M, HPMC-AS-H, or Eudragit L.

5. A pharmaceutical composition of a compound represented by Formula (I) 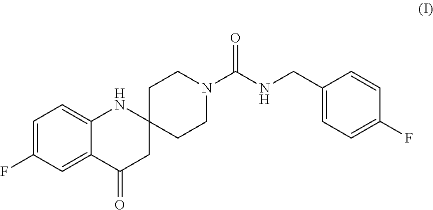 [SMILES: O=C1CC2(CCN(C(=O)NCc3ccc(F)cc3)CC2)Nc2ccc(F)cc21]
comprising:
(i) an intragranular blend, wherein the intragranular blend comprises:
(a) an amorphous spray granulate comprising:
(i) the compound of Formula (I), wherein the compound is present in amorphous form;
(ii) a polymer;
(b) a suspending agent;
(c) a carrier;
(ii) an extragranular blend, wherein the extragranular blend comprises:
(d) a filler;
(e) disintegrant;
(f) a glidant; and
(g) a lubricant.

6. The pharmaceutical composition of claim 5 comprising:
(i) an intragranular blend, wherein the intragranular blend comprises:
(a) an amorphous spray granulate comprising:
(i) the compound of Formula (I), wherein the compound is present in amorphous form, in an amount of about 5 wt % to 20 wt %;
(ii) a polymer in an amount of about 10 wt % to 60 wt %;
(b) a suspending agent in an amount of about 0.5 wt % to 2.0 wt %;
(c) a carrier in an amount of about 20 wt % to 80 wt %;
(ii) an extragranular blend, wherein the extragranular blend comprises:
(d) a filler in an amount of about 10 wt % to 40 wt %;
(e) a disintegrant in an amount of about 0 wt % to 5 wt %;
(f) a glidant in an amount of about 0.5 wt % to 2.0 wt %; and
(g) a lubricant in an amount of about 0.5 wt % to 3.0 wt %.

7. The pharmaceutical composition of claim 6 comprising:
(i) an intragranular blend, wherein the intragranular blend comprises:
(a) an amorphous spray granulate comprising:
(i) the compound of Formula (I), wherein the compound is present in amorphous form, in an amount of 11.9 wt %;
(ii) a polymer in an amount of about 26.6 wt %;;
(b) a suspending agent in an amount of about 1.3 wt %;
(c) a carrier in an amount of about 30.3 wt %;
(ii) an extragranular blend, wherein the extragranular blend comprises:
(d) a filler in an amount of about 24.6 wt %;
(e) a disintegrant in an amount of about 2.8 wt %;
(f) a glidant in an amount of about 1.0 wt %; and
(g) a lubricant in an amount of about 1.5 wt %.

8. The pharmaceutical composition of claim 5, wherein the polymer is hydroxypropyl methyl cellulose acetate succinate (HPMC-AS).

9. The pharmaceutical composition of claim 8, wherein the hydroxypropyl methyl cellulose acetate succinate is selected from hydroxypropyl methyl cellulose acetate succinate L grade (HPMC-AS-L), hydroxypropyl methyl cellulose acetate succinate M grade (HPMC-AS-M), and hydroxypropyl methyl cellulose acetate succinate H grade (HPMC-AS-H).

10. The pharmaceutical composition of claim 7, wherein the suspending agent is silicon dioxide.

11. The pharmaceutical composition of claim 7, wherein the carrier is lactose.

12. The pharmaceutical composition of claim 7, wherein the filler is lactose, microcrystalline cellulose, low substituted hydroxypropyl cellulose (L-HPC), or a combination thereof.

13. The pharmaceutical composition of claim 7, wherein the disintegrant is croscarmellose sodium.

14. The pharmaceutical composition of claim 7, wherein the glidant is silicon dioxide.

15. The pharmaceutical composition of claim 7, wherein the lubricant is sodium stearyl fumarate.

16. The pharmaceutical composition of claim 7, comprising:
(i) an intragranular blend, wherein the intragranular blend comprises:
(a) an amorphous spray granulate comprising:
(i) the compound of Formula (I), wherein the compound is present in amorphous form, in an amount of 11.9 wt %;
(ii) hydroxypropyl methyl cellulose acetate succinate in an amount of about 26.6 wt %;
(b) silicon dioxide in an amount of about 1.3 wt %;
(c) lactose in an amount of about 30.3 wt %;
(ii) an extragranular blend, wherein the extragranular blend comprises:
(d) lactose and microcrystalline cellulose in an amount of about 24.6 wt %;
(e) croscarmellose sodium in an amount of about 2.8 wt %;
(f) silicon dioxide in an amount of about 1.0 wt %; and
(g) sodium stearyl fumarate in an amount of about 1.5 wt %.

17. The pharmaceutically acceptable composition of claim 1, wherein the composition is in the form of a capsule or tablet.

18. A process of manufacturing a pharmaceutical composition according to claim 5 comprising:
(i) preparation of an amorphous spray granulation, comprising:
(a) suspending compound of Formula (I), a polymer and a suspending agent into an organic solution of acetone containing water;
(b) mixing the suspension of (a) to form a dispersion of dissolved compound of Formula (I);
(c) spraying the dispersion of (b) onto a carrier in a fluid bed dryer; to form an amorphous spray granulate;
(ii) preparing an extragranular phase, wherein the extragranular phase comprises:
(d) a filler;
(e) a disintegrant;
(f) a glidant; and
(g) a lubricant
(iii) blending the amorphous spray granulate (i) and the extrangranular phase (ii) to form a final blend.

19. The process of manufacturing a pharmaceutical composition according to claim 18, wherein the composition is filled into a capsule.

20. The process of manufacturing a pharmaceutical composition according to claim 18, wherein the composition is compacted into a tablet.

21. A method of treating a disease selected from the group consisting of gastrointestinal stromal tumors (GIST), NF-1-deficient gastrointestinal stromal tumors, succinate dehydrogenase (SDH)-deficient gastrointestinal stromal tumors, KIT driven gastrointestinal stromal tumors, PDGFRA driven gastrointestinal stromal tumors, melanoma, acute myeloid leukemia, germ cell tumors of the seminoma or dysgerminoma, mastocytosis, mast cell leukemia, lung adenocarcinoma, squamous cell lung cancer, glioblastoma, glioma, pediatric glioma, astrocytomas, sarcomas, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, lymphoblastic T-cell lymphoma, liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), myelodysplastic syndrome (MDS),and non-small cell lung cancer, comprising administering to a patient in need thereof a therapeutically effective amount of the composition of claim 1.

22. The method of claim 21, wherein the disease is non-small cell lung cancer (NSCLC).

23. The use of the composition of claim 1 for the preparation of a medicament for the treatment of a disease selected from the group consisting of gastrointestinal stromal tumors (GIST), NF-1-deficient gastrointestinal stromal tumors, succinate dehydrogenase (SDH)-deficient gastrointestinal stromal tumors, KIT driven gastrointestinal stromal tumors, PDGFRA driven gastrointestinal stromal tumors, melanoma, acute myeloid leukemia, germ cell tumors of the seminoma or dysgerminoma, mastocytosis, mast cell leukemia, lung adenocarcinoma, squamous cell lung cancer, glioblastoma, glioma, pediatric glioma, astrocytomas, sarcomas, malignant peripheral nerve sheath sarcoma, intimal sarcomas, hypereosinophilic syndrome, idiopathic hypereosinophilic syndrome, chronic eosinophilic leukemia, eosinophilia-associated acute myeloid leukemia, lymphoblastic T-cell lymphoma, liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), myelodysplastic syndrome (MDS), and non-small cell lung cancer.

24. The use of claim 23, wherein the disease is non-small cell lung cancer (NSCLC).

25. A pharmaceutical composition of a compound represented by Formula (I) 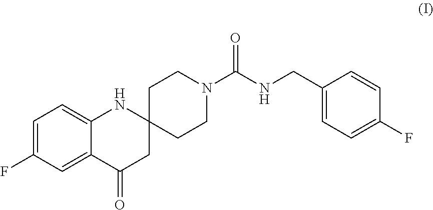 [SMILES: O=C1CC2(CCN(C(=O)NCc3ccc(F)cc3)CC2)Nc2ccc(F)cc21]
comprising:
(i) a nanosized crystalline spray granulate, comprising:
(a) the compound of Formula (I), in crystalline Form A, wherein the crystals have been nano sized,
(b) a polymer,
(c) a surfactant;
(d) a carrier;
(ii) an extragranular blend, wherein the extragranular blend comprises:
(e) one or more fillers;
(f) a disintegrant;
(g) a glidant; and
(h) a lubricant.

26. The pharmaceutical composition of claim 25 comprising:
(i) a nanosized crystalline spray granulate, wherein the crystalline nanospray granulate comprises:
(a) the compound of Formula (I), wherein the compound of Formula (I) is nanosized crystalline Form A in an amount of about 5 wt % to about 20 wt %,
(b) a polymer in an amount of about 5 wt % to 20 wt %,
(c) a surfactant in an amount of about 0.1 wt % to 1.0 wt %;
(d) a carrier in an amount of about 20 wt % to 80 wt %;
(ii) an extragranular blend, wherein the extragranular blend comprises:
(e) one or more fillers in an amount of about 25 wt % to 50 wt %;
(f) a disintegrant in an amount of about 2 wt % to 10 wt %;
(g) a glidant in an amount of about 0.5 wt % to 2.0 wt %; and
(h) a lubricant in an amount of about 0.5 wt % to 2.0 wt %.

27. The pharmaceutical composition of claim 26 comprising:
(i) a nanosized crystalline spray granulate, wherein the crystalline nanospray granulate comprises:
(a) the compound of Formula (I), wherein the compound of Formula (I) is nanosized crystalline Form A in an amount of 12.5 wt %,
(b) a polymer in an amount of about 8.34%,
(c) a surfactant in an amount of about 0.25 wt %;
(d) a carrier in an amount of about 28.9 wt %;
(ii) an extragranular blend, wherein the extragranular blend comprises:
(e) one or more fillers in an amount of about 40 wt %;
(f) a disintegrant in an amount of about 6 wt %;
(g) a glidant in an amount of about 1.5 wt %; and
(h) a lubricant in an amount of about 1.5 wt %.

28. The pharmaceutical composition of claim 5, wherein the polymer is povidone or copovidone.

29. The pharmaceutical mpostiG of claim 28, wherein povidone is PVP K30.

30. The pharmaceutical composition of claim 27 comprising:
(i) a crystalline nanospray granulate, wherein the crystalline nanospray granulate comprises:
(a) the compound of Formula (I), wherein the compound of Formula (I) is nanosized crystalline Form A in an amount of 12.5 wt %,
(b) povidone in an amount of about 8.34%,
(c) sodium lauryl sulfate in an amount of about 0.25 wt %;
(d) a lactose carrier in an amount of about 28.9 wt %;
(ii) an extragranular blend, wherein the extragranular blend comprises:
(e) lactose and microcrystalline cellulose in an amount of about 40 wt %;
(f) croscarmellose sodium in an amount of about 6 wt %;
(g) silicon dioxide in an amount of about 1.5 wt %; and
(h) sodium steraryl fumarate in an amount of about 1.5 wt %.

31. A pharmaceutical mpestiG of claim 25, wherein the crystals of the compound of Formula (I), crystalline Form A, have a median particle diameter (D50) of about 150 to 250 nm.

32. A process for preparing the pharmaceutical composition according to claim 25, said process comprising the steps of:
(i) mixing a mixture comprising the compound of Formula (I), in crystalline Form A, wherein the crystals have been nano sized, a polymer, and a surfactant in a liquid medium, and
(ii) adding said mixture to the carrier to form dry granulate.

33. The process according to claim 32, wherein step (i) is performed in a wet milling chamber.

34. The process according to claim 32, wherein the liquid medium is an aqueous solution.

35. The process according to claim 32, wherein the mixture of step (i) is dispersed onto the carrier and dried to form granulate.

36. The process according to claim 32, wherein the process further comprises preparing the final dosage form by blending the granulate resulting from step (ii) with with extragranular phase, wherein the extragranular phase comprises one or more: filler; disintegrant; glidant; lubricant.

37. The process according to claim 36, wherein the final dosage form is encapsulated or tableted.

38. The process according to claim 37, wherein the final dosage form is tableted and the resulting tablet is further film coated.

39. A process for preparing a suspension comprising mixing the Compound of Formula (I), or a pharmaceutically acceptable salt thereof, or a free form thereof, at least one polymer, and optionally a surfactant, with a liquid medium.

40. The process according to claim 39, wherein the suspension is subjected to wet milling in order to reduce the size of crystals of Compound of Formula (I).

41. The suspension according to claim 40 wherein the median particle diameter (D50) of crystals of Compound of Formula (I) in said suspension is about 100 nm to 500 nm.

42. The suspension according to claim 41 wherein the median particle diameter (D50) of crystals of Compound of Formula (I) in said suspension is about 150 nm to 250 nm.
